(19) United States
(12) Patent Application Publication
(10) Pub. No.: US 2017/0312057 A1
(43) Pub. Date: Nov. 2, 2017

(54) TOOTH IMPLANTATION TECHNIQUE AND A SUBPERIOSTEAL IMPLANT MANUFACTURING METHOD FOR THE SOLUTION OF EXTREME ATROPHY OF A TOOTH BONE

(71) Applicant: Jung-Chuan CHENG, New Taipei City (TW)

(72) Inventor: Jung-Chuan CHENG, New Taipei City (TW)

(21) Appl. No.: 15/143,129

(22) Filed: Apr. 29, 2016

(51) Int. Cl. A61C 13/00; A61C 8/00; A61B 6/02; A61B 6/14; A61C 13/34

(52) U.S. Cl. CPC A61C 13/0004; A61C 13/34; A61C 13/0028; A61C 13/0019; A61B 6/02; A61C 8/0012; A61B 6/14; A61C 8/0031

(57) ABSTRACT

A tooth implantation technique and a subperiosteal implant manufacturing method for the solution of extreme atrophy of a tooth bone are disclosed, primarily using steps of tomography, data transformation, software design, 3D printing of a physical model, making a subperiosteal implant wax form, wax form scanning and design, and manufacturing with a CNC milling machine, so as to make a subperiosteal implant that fits with a patient, thereby improving the shortcomings in a conventional manufacturing procedure of an implant.

### BACKGROUND OF THE INVENTION

### a) Field of the Invention

The present invention relates to a tooth implantation technique and a subperiosteal implant manufacturing method for the solution of extreme atrophy of a tooth bone.

### b) Description of the Prior Art

It is known that when a tooth bone in the oral cavity of an edentulous patient is extremely atrophied (reaching level 4 in the bone mass classification that the alveolar bone has been absorbed completely and the basal bone has been absorbed, leaving behind a very little amount), it will be unable to take a conventional implant reconstruction procedure and normally two kinds of medical treatment plans will be used.

For the first medical treatment plan, Prof. Branemark (also a brand name of an implant) came up with a Zygomal implantation plan in which a long implant structure is penetrated and planted into the zygomatic bone from the oral cavity, and a denture is reconstructed by plural implants that are extended into the oral cavity. However, as most of the edentulous patients who are suffered from the extreme atrophy of the alveolar bone in the oral cavity are elderly people, their physical condition is weak and if the Zygoma implantation plan is used, they have to be admitted in a hospital for the operation in general anesthesia as the wound is large. Therefore, the risk is relatively higher if an elder receives this kind of major operation and it will be a heavy burden to the patient and his or her family both physically and mentally.

For the second medical treatment plan, in about 50-60 years ago, the dental professionals have used a subperiosteal implantation plan. This plan includes two operations. In the first operation, the gingiva of a patient is cut open and flapped to get a bone model and perform an implant design. After the implant design has been accomplished and the wound of the first operation has been recovered (in about 1-2 months), the patient's gingiva has to be cut open and flapped one more time to embed the implant. The shortcoming is that when getting the bone model, it is not easy to control the blood volume, which will affect the precision of the bone model. In addition, the patient has to deal with the fear of receiving a second operation after waiting for a period of time allowing the wound of gingiva to be recovered, as taking two large-incision flap operations will be a great suffering to the patient. Furthermore, the implant of this plan is made of titanium alloy manually by casting. As titanium is poor in fluidity, the implant cannot be always made successfully and the failure rate is extremely high.

Due to the shortcomings in the two abovementioned medical treatment plans, they have not yet been applied truly effectively for a long time to the clinical treatment to an edentulous patient who suffers from the extreme atrophy of the alveolar bone in the oral cavity. Accordingly, for the shortcomings in the abovementioned plans, the present invention employs a high-end technology in modern medical imaging, CAD/CAM (Computer Aided Design/Computer Aided Manufacturing) software and hardware, 3D print and CNC (Computer Numerically Controlled) engraving equipment to make a disruptive innovation in the manufacturing process, thereby benefiting the edentulous patients who suffer from the extreme atrophy of the alveolar bone.

### SUMMARY OF THE INVENTION

The primary object of the present invention is to provide a tooth implantation technique and a subperiosteal implant manufacturing method for the solution of extreme atrophy of a tooth bone, allowing a patient to free from the old-style operation that an implant is penetrated into the zygomatic bone, the gingiva is flapped twice and the patient has to face the high risk. In addition, the present invention can remove the implant manufacturing process that is poor in precision and provided with a high failure rate, in order to truly achieve the highest efficient medical treatment that is safe, saves time and is less painful.

To achieve the abovementioned object, the present invention discloses a tooth implantation technique and a subperiosteal implant manufacturing method for the solution of extreme atrophy of a tooth bone, including: (a) conducting tomography to the part where the tooth bone is extremely atrophied to get an image file of the tooth bone; (b) using design software to transform the tooth bone image file into a digital tooth bone image file that can be edited by the design software; (c) using the design software to design and edit the digital tooth bone image file, making a tooth bone physical model file; (d) using 3D print equipment to make a tooth bone physical model from the tooth bone physical model file; (e) using the tooth bone physical model as a substrate to make a subperiosteal implant wax form; (f) using stereoscopic scanning equipment to scan the subperiosteal implant wax form, and saving the results as a subperiosteal implant wax form file to be edited by the design software; (g) using the design software to edit the subperiosteal implant wax form file, forming a subperiosteal implant file that can be accessed for production by manufacturing equipment; (h) using a CNC milling machine to conduct final production, grinding and cleaning, based upon the subperiosteal implant file, in order to form a physical subperiosteal implant.

According to an embodiment of the present invention, the present invention further includes step (d1), in which based upon the tooth bone physical model, a replica model is duped with gypsum, acting as a replacement to the substrate in step (e).

According to an embodiment of the present invention, the tooth bone physical model is a plastic RP (Rapid Prototyping) model.

According to an embodiment of the present invention, the tomography is X-ray stereoscopic photography.

According to an embodiment of the present invention, the subperiosteal implant is made of Ti-6Al-4V (titanium alloy).

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a flow diagram of a preferred embodiment of the present invention.

FIG. 1A shows a flow diagram of duping with gypsum in a preferred embodiment of the present invention.

FIG. 2 shows a schematic view of a file of tooth bone physical model which is transformed and edited by design software of the present invention.

FIG. 3 shows a schematic view of a tooth bone physical model which is manufactured by 3D print of the present invention.

FIG. 4 shows a schematic view of a subperiosteal implant wax form which is made using the tooth bone physical model as a substrate of the present invention.

FIG. 5 shows a schematic view of a file of subperiosteal implant wax form which is scanned and saved of the present invention.

FIG. 6 shows a schematic view of a subperiosteal implant which is made by a milling machine of the present invention.

### DETAILED DESCRIPTION OF THE PREFERRED EMBODIMENTS

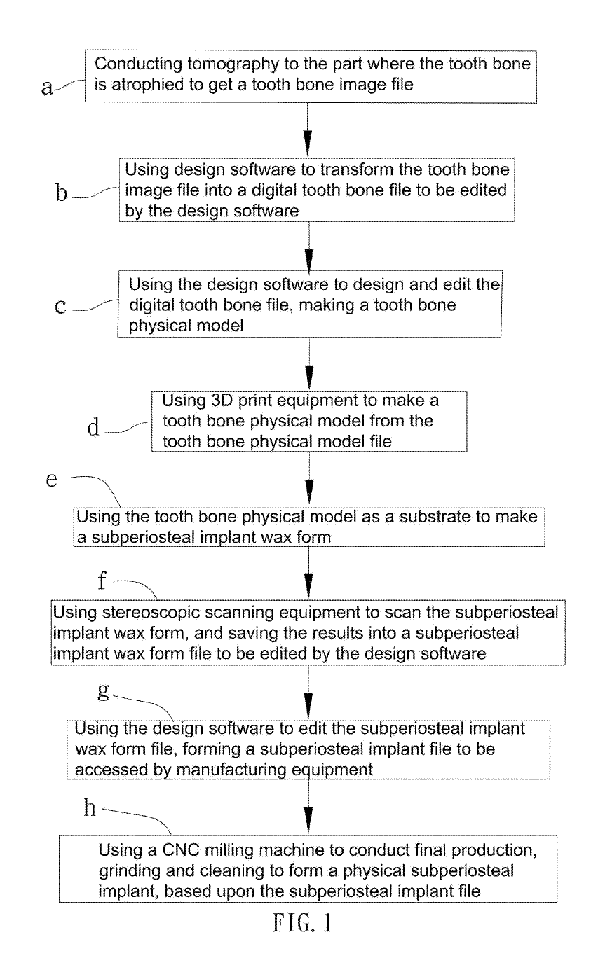

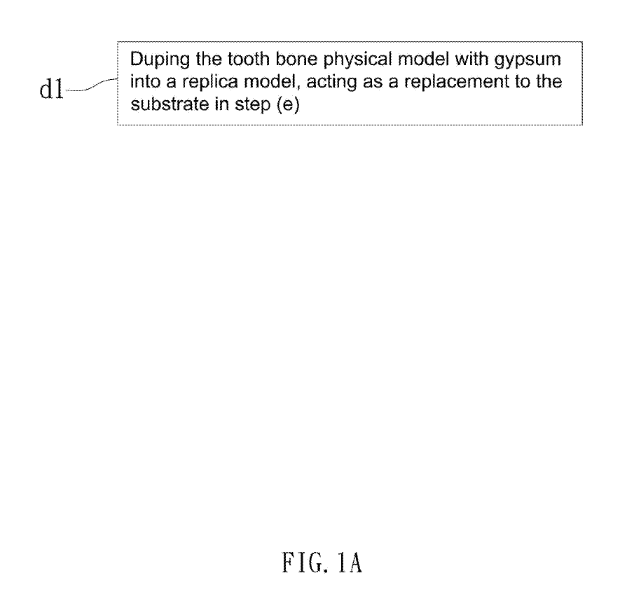

FIG. 1 and FIG. 1A show a flow diagram of a preferred embodiment of the present invention and a flow diagram of duping with gypsum in a preferred embodiment of the present invention. The present invention discloses a tooth implantation technique and a subperiosteal implant manufacturing method for the solution of extreme atrophy of a tooth bone, including following steps: (a) conducting tomography (X-ray stereoscopic photography) to the part where the tooth bone is extremely atrophied to get an image file of the tooth bone (the tomography step); (b) using design software to transform the tooth bone image file into a digital tooth bone image file that can be edited by the design software (the data transformation step); (c) using the design software to design and edit the digital tooth bone image file, making a tooth bone physical model file (the software design step); (d) using 3D print equipment to make a tooth bone physical model (a plastic RP model) from the tooth bone physical model file (the step of 3D printing of a physical model); (e) using the tooth bone physical model as a substrate to make a subperiosteal implant wax form (the step of making a subperiosteal implant wax form); (f) using stereoscopic scanning equipment to scan the subperiosteal implant wax form, and saving the results as a subperiosteal implant wax form file to be edited by the design software (the step of wax form scanning and design); (g) using the design software to edit the subperiosteal implant wax form file, forming a subperiosteal implant file that can be accessed for production by manufacturing equipment; (h) using a CNC milling machine to conduct final production, grinding and cleaning, based upon the subperiosteal implant file, in order to form a physical subperiosteal implant (Ti-6Al-4V [titanium alloy]) (the step of manufacturing by a CNC milling machine). The present invention further includes step (d1), in which based upon the tooth bone physical model, a replica model is duped with gypsum, acting as a replacement to the substrate in step (e).

The hardware used in step (a) above is GE Light Speed VCT 64 Slice CT, with the specification of Slice 0.625 mm, Time 60 sec, Mx 240 8.0 MHU Tube, Oil/Air Tube Cooling, and 3000 Image Series (Direct 3D).

The software used in step (c) above are: (1) Analyze PC 3.0 Mayo Foundation (2) Mimics 6.3 Materialise (3) Power Solution Delcam (4) Project 3510 SD&HD (5) 3D Systems (RP).

The hardware used in step (d) above are: (1) SLA-5000 3D System (2) Thermojet 3D Printer and 3D System.

The hardware used in step (f) above is Dental Scanner (DS200), and the software used in step (g) above is Exocad.

The hardware used in step (h) above is a TDS digital 5-axis dental milling machine (ME-300HP).

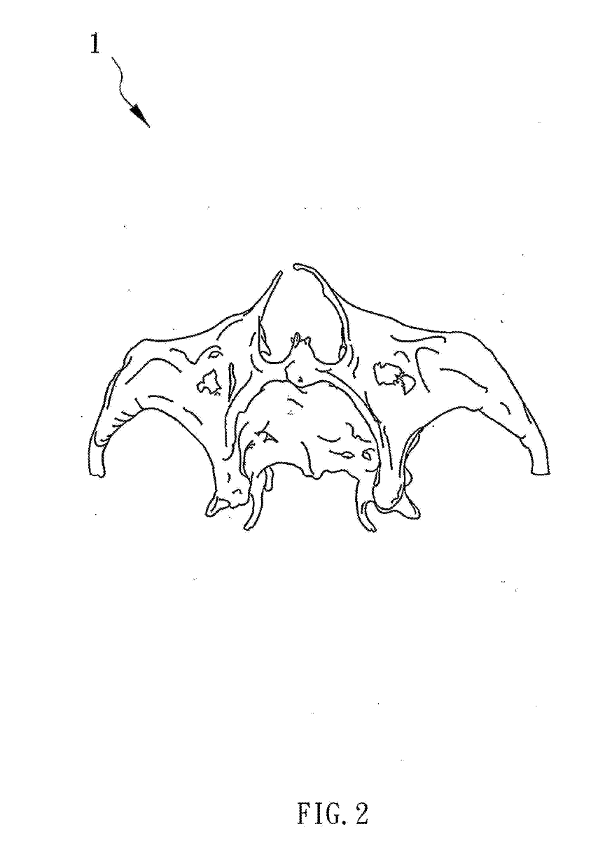

Referring to FIGS. 2 to 6 together, it shows a schematic view of a digital tooth bone physical model file which is transformed and edited by design software, a schematic view of a tooth bone physical model which is manufactured by 3D print, a schematic view of a subperiosteal implant wax form which is made using the tooth bone physical model as a substrate, a schematic view of a subperiosteal implant wax form file which is scanned and saved, and a schematic view of a subperiosteal implant which is made by a milling machine.

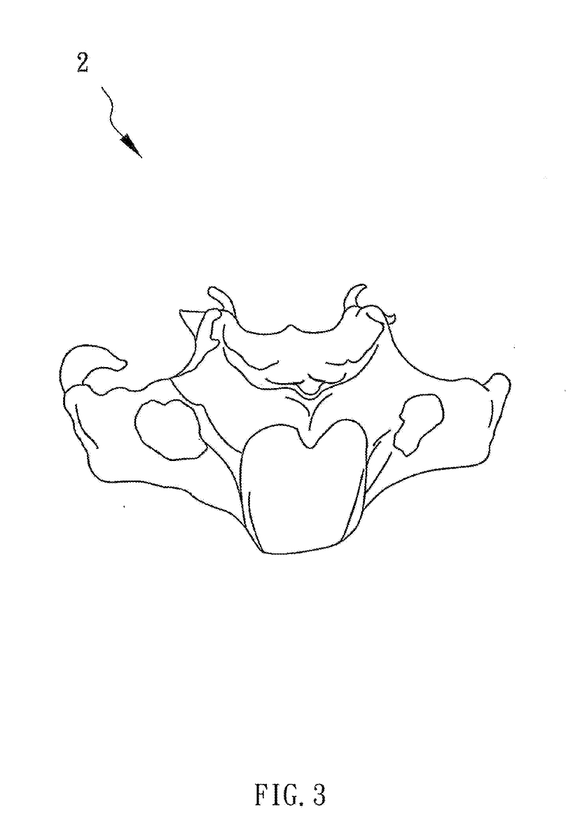

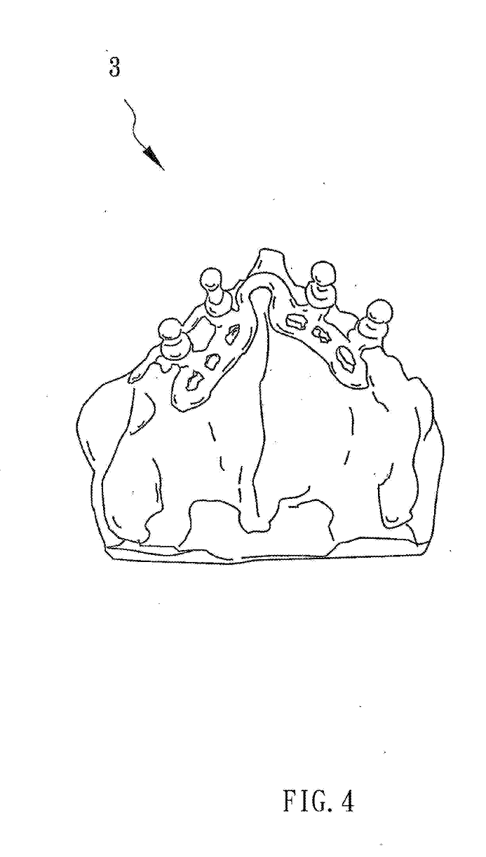

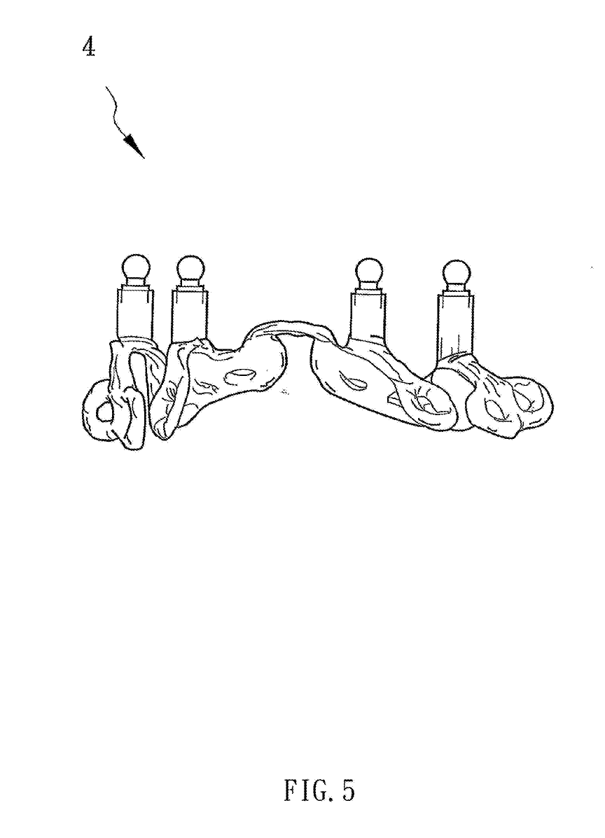

Before making a subperiosteal implant for a patient of tooth bone atrophy, the part where the tooth bone is atrophied should be first conducted with tomography by X-ray stereoscopic photography to get a tooth bone image file. As this tooth bone image file is an image file, it needs to be transformed into a digital tooth bone file 1. This digital tooth bone file 1 provides for access, editing and design by design software (as shown in FIG. 2), and for the post-edited digital tooth bone file 1, the related coding and path can be accessed by equipment for making a physical model (3D print equipment). The 3D print equipment accesses the related coding in the digital tooth bone file 1 from the path and makes a tooth bone physical model 2 (a plastic RP model, as shown in FIG. 3). From the accomplished tooth bone physical model, the actual condition of the tooth bone atrophy to the patient can be clearly known. At this time, in order to allow the subperiosteal implant to be fixed on a physical tooth bone perfectly, the tooth bone physical model should be used as a substrate to make a subperiosteal implant wax form 3 (as shown in FIG. 4). This subperiosteal implant wax form 3 is not a template for final production, and should be scanned by stereoscopic scanning equipment. This step scans the prepared subperiosteal implant wax form 3 with stereoscopic scanning equipment to result in a subperiosteal implant wax form file 4 that can be edited by the design software. The subperiosteal implant wax form file 4 is edited, designed and saved as a subperiosteal implant wax file 4 that can be accessed by manufacturing equipment (as shown in FIG. 5). Then, using the design software to edit and modify various parameters, such as angle, curvature, length and width, of the subperiosteal implant wax form file, the final product of the subperiosteal implant can fit better on the patient's actual tooth bone that the patient will not feel uncomfortable. It is worth of mentioning that the present invention further includes step (d1), in which based upon the tooth bone physical model, a replica model is duped with gypsum to act as a replacement to the substrate in step (e).

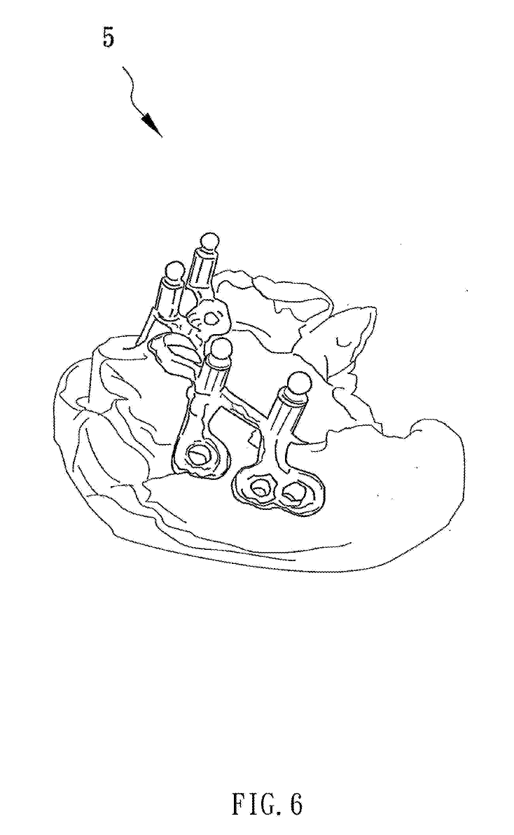

After accomplishing the abovementioned tomography step, data transformation step, software design step, step of 3D printing of a physical model, step of making a subperiosteal implant wax form, and step of wax form scanning and design, the final subperiosteal implant form can be ascertained. Finally, step (h) is used to accomplish manufacturing by a CNC milling machine. The CNC milling machine (5-axis CNC precision engraving machine) accesses from the path the related coding in the subperiosteal implant file planned by the design software, and engraves medical-use Ti-6Al-4V (titanium alloy) into a subperiosteal implant 5 (as shown in FIG. 6) in accordance with the coding instructions.

Finally, the accomplished subperiosteal implant is implanted into subperiosteum in an oral cavity through only one subperiosteal implantation operation to a patient.

According to the present invention, the tooth implantation technique and the subperiosteal implant manufacturing method for the solution of extreme atrophy of a tooth bone allow a patient to free from the pain and high risk in the old-style operation that the tooth implant is penetrated into the zygomatic bone and the gingiva is flapped twice. In addition, the tooth implant manufacturing procedure that is poor in precision and is provided with a high failure rate will not be used, which truly achieves the highest efficient medical treatment that is safe, saves time and is less painful.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A tooth implantation technique and a subperiosteal implant manufacturing method for solution of extreme atrophy of a tooth bone, comprising following steps:
(a) conducting tomography to a part where the tooth bone is extremely atrophied to get an image file of the tooth bone;
(b) using design software to transform the tooth bone image file into a digital tooth bone image file to be edited by the design software;
(c) using the design software to design and edit the digital tooth bone image file, making a tooth bone physical model file;
(d) using 3D print equipment to make a tooth bone physical model from the tooth bone physical model file;
(e) using the tooth bone physical model as a substrate to make a subperiosteal implant wax form;
(f) using stereoscopic scanning equipment to scan the subperiosteal implant wax form, and saving the results as a subperiosteal implant wax form file to be edited by the design software;
(g) using the design software to edit the subperiosteal implant wax form file, forming a subperiosteal implant file to be accessed for production by manufacturing equipment;
(h) using a CNC (Computer Numerically Controlled) milling machine conduct final production, grinding and cleaning, based upon the subperiosteal implant file, in order to form a physical subperiosteal implant.

2. The tooth implantation technique and the subperiosteal implant manufacturing method for solution of extreme atrophy of a tooth bone, according to claim 1, further including step (d1), in which based upon the tooth bone physical model, a replica model is duped with gypsum, acting as a replacement to the substrate in step (e).

3. The tooth implantation technique and the subperiosteal implant manufacturing method for solution of extreme atrophy of a tooth bone, according to claim 1, wherein the tooth bone physical model is a plastic RP (Rapid Prototyping) model.

4. The tooth implantation technique and the subperiosteal implant manufacturing method for solution of extreme atrophy of a tooth bone, according to claim 1, wherein the tomography is X-ray stereoscopic photography.

5. The tooth implantation technique and the subperiosteal implant manufacturing method for solution of extreme atrophy of a tooth bone, according to claim 1, wherein the subperiosteal implant is made of Ti-6Al-V (titanium alloy).
